(19) United States
(12) Patent Application Publication
(10) Pub. No.: US 2026/0365630 A1
(43) Pub. Date: Nov. 19, 2026

(54) PROCESS FOR TRAPPING THIOCARBONYL FLUORIDE

(71) Applicants: CENTRE NATIONAL DE LA RECHERCHE SCIENTIFIQUE, Paris (FR); INSTITUT NATIONAL DES SCIENCES APPLIQUEES DE ROUEN NORMANDIE, Saint-Etienne-du-Rouvray (FR); UNIVERSITÉ DE ROUEN NORMANDIE, Mont Saint-Aignan (FR)

(72) Inventors: Tatiana BESSET, Mont Saint Aignan Cedex (FR); Thomas CASTANHEIRO, Malaunay (FR); Enzo NOBILE, Massy (FR); Mathieu ARRIBAT, Paulhan (FR)

(21) Appl. No.: 19/487,270

(22) PCT Filed: May 30, 2024
(86) PCT No.: PCT/EP2024/064959

§ 371 Date: Nov. 24, 2025

(30) Foreign Application Priority Data
Jun. 1, 2023 (EP) 23305869.2

(51) Int. Cl. C07D 277/68; C07C 329/00; C07C 333/00; C07D 277/74; C07D 277/82; C07D 295/03; C07D 295/033; C07D 417/12

(52) U.S. Cl. CPC C07D 277/68; C07C 329/00; C07C 333/00; C07D 277/74; C07D 277/82; C07D 295/03; C07D 295/033; C07D 417/12

(57) ABSTRACT

The present application relates to a process for trapping SCF₂ including the following steps:
a step of reacting a compound A-SCF₃ with a nucleophile, in the presence of a metallic complex including nickel, palladium or rhodium(I), a ligand, and a solvent,
A being selected from the group consisting of: (C₆-C₁₀)aryl groups, heteroaryl groups, and a vinyl compound,
   said step leading to the formation of a compound AH or a compound AZ and SCF₂, wherein Z is a hydrocarbon group, optionally including at least one heteroatom such as S, N or O, and/or at least one atom other than C or H, such as Se, Si, B, or P, and
a step of reacting SCF₂ with a compound RH, R being a hydrocarbon group, including optionally at least one heteroatom,
for obtaining a compound having the formula R—C(═S)—F.

The present invention concerns a process for trapping thiocarbonyl fluoride or analogues thereof.

In a society concerned about the environment, earth and aquatic systems pollution and circular economy, development of new technologies to meet such objectives is nowadays a real challenge and a hot topic across the whole chemical industry.

The field of organofluorine chemistry is unavoidable and fluorinated compounds are highly represented in many fields such as materials science, pharmaceutical and agrochemical industries. Indeed, the incorporation of a fluorine atom or a fluorinated unit can modify the physico-chemical properties of organic molecules in a significant way explaining the interest of the scientific community regarding this research area. However, the prevalence of these compounds raises the question of their fate and degradation. Recently, there has been a strong awareness from the scientific community and society regarding chlorofluorocarbons (CFCs), hydrofluorocarbons (HFCs) and per- and poly-fluoroalkyl substances (PFAs). It is now important to go even further and to look at the fate of emerging fluorinated groups that have become essential, such as derivatives containing a SCF₃ unit (eg. Toltrazuril and Fipronil).

In this context, the development of tools allowing to valorize these fluorinated derivatives, which, once used, are then considered as waste by converting them into compounds with high added value would have an undeniable ecological impact and allow for cost reduction. It is therefore essential to develop innovative tools to meet this challenge and to remove this synthetic lock.

There is currently no technology allowing to selectively cleave C(sp²)-SRf bonds on (hetero) aromatic derivatives or vinyl positions or C(sp³)-SRf bonds and any progress would have a major impact from a scientific and environmental point of view.

N. Barbero et al., Organic Letters, 2012, 14, 796-799 (Supporting Information, S1-S78) relates to catalytic reductive cleavage of unactivated C—SMe bonds. As shown on page 797, left column, last paragraph, the SMe group, namely an electron-donating group on the sulfur atom, was reported as the most efficient in the reductive cleavage of C—S bond. The optimization process reported in Tables 1 to 6 of the supporting information (p. S3 to S5) was carried out for this specific thioether only. Table 7 (p. S5) of this document confirms that SMe group raises the highest yield with respect to cleavage of the C—S bond, while electron-attracting groups, such as Ac and CONMe₂, were inefficient.

Surprisingly, it has now been found that it was possible to selectively cleave C—S bonds and analogues of such bonds on organic molecules in the presence of an electron-attracting fluorinated group, such as CF₃ or analogous fluorine-containing groups.

With a view to the circular economy, which is nowadays an essential part of industrial purposes, notably through the 3R logic (Reduce, Reuse, Recycle), there is a need of reusing the fluorinated residue released in the course of the de-fluorination reaction.

One aim of the present invention is thus to provide a process for trapping a fluorinated residue, thiocarbonyl fluoride (SCF₂), SCF₃⁻ or F⁻.

The present invention relates to a process for trapping SCF₂ comprising the following steps:
a step of reacting a compound having the formula (I)

A-SCF₃ (I)
with a nucleophile, in the presence of a metallic complex comprising nickel, palladium or rhodium(I), a ligand, and a solvent,
A being selected from the group consisting of:
   (C₆-C₁₀)aryl groups,
   said aryl groups being optionally substituted with at least one substituent preferably selected from the group consisting of: halogen, (C₁-C₆)alkyl, (C₁-C₆)alkoxy, —CN, —CF₃, —OCF₃, —NRₐR_{b}, —SiRₐR_{b}R_{c}, —BRₐR_{b}, (C₂-C₆)alkenyl, (C₂-C₆)alkynyl, —C(═O)—NRₐR_{b}, and —C(═O)—O(C₁-C₆)alkyl;
   Rₐ and R_{b} being independently from each other H or a (C₁-C₆)alkyl group; R_{c} being a (C₁-C₆)alkyl group;
   heteroaryl groups comprising from 5 to 10 atoms and including at least one heteroatom selected from O, N, and S,
   said heteroaryl groups being optionally substituted with at least one substituent preferably selected from: halogen, (C₁-C₆)alkyl, (C₁-C₆)alkoxy, —CN, —CF₃, —OCF₃, —NRₐR_{b}, —Si(R_{c})₃, —BRₐR_{b}, (C₂-C₆)alkenyl, (C₂-C₆)alkynyl, —C(═O)—NRₐR_{b}, and —C(═O)—O(C₁-C₆)alkyl, Rₐ, R_{b} and R_{c} being as defined above; and
   a vinyl compound having the following formula (III):

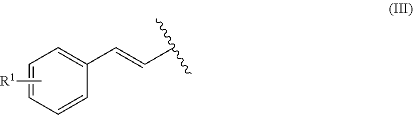

R¹ being selected from the group consisting of: halogen, (C₁-C₆)alkyl, (C₁-C₆)alkoxy, —CN, —CF₃, —OCF₃, —NRₐR_{b}, —Si(R_{c})₃, —BRₐR_{b}, (C₂-C₆)alkenyl, (C₂-C₆)alkynyl, —C(═O)—NRₐR_{b}, and —C(═O)—O(C₁-C₆)alkyl, Rₐ, R_{b} and R_{c} being as defined above; and
said step leading to the formation of a compound AH or a compound AZ and SCF₂, wherein Z is a hydrocarbon group, optionally including at least one heteroatom such as S, N or O, and/or at least one atom other than C or H, such as Se, Si, B, or P,
   and
   a step of reacting SCF₂ obtained at the previous step with a compound RH, R being a hydrocarbon group, comprising optionally at least one heteroatom,
   for obtaining a compound having the formula R—C(═S)—F.

In particular, the present invention relates to a process for trapping SCF₂ comprising the following steps:
a step of reacting a compound having the formula (I)

A-SCF₃ (I)
with a nucleophile, in the presence of a metallic complex comprising nickel, a ligand, and a solvent,
A being selected from the group consisting of:
   (C₆-C₁₀)aryl groups,
   said aryl groups being optionally substituted with at least one substituent preferably selected from the group consisting of: halogen, (C₁-C₆)alkyl, (C₁-C₆)alkoxy, —CN, —CF₃, —OCF₃, —NRₐR_{b}, —SiRₐR_{b}R_{c}, —BRₐR_{b}, (C₂-C₆)alkenyl, (C₂-C₆)alkynyl, —C(═O)—NRₐR_{b}, and —C(═O)—O(C₁-C₆)alkyl;
   Rₐ and R_{b} being independently from each other H or a (C₁-C₆)alkyl group;
   R_{c} being a (C₁-C₆)alkyl group;
   heteroaryl groups comprising from 5 to 10 atoms and including at least one heteroatom selected from O, N, and S,
   said heteroaryl groups being optionally substituted with at least one substituent preferably selected from: halogen, (C₁-C₆)alkyl, (C₁-C₆)alkoxy, —CN, —CF₃, —OCF₃, —NRₐR_{b}, —Si(R_{c})₃, —BRₐR_{b}, (C₂-C₆)alkenyl, (C₂-C₆)alkynyl, —C(═O)—NRₐR_{b}, and —C(═O)—O(C₁-C₆)alkyl, Rₐ, R_{b} and R_{c} being as defined above; and
   a vinyl compound having the following formula (III):

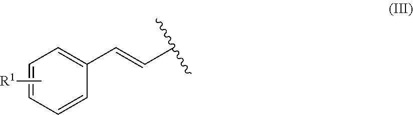

R¹ being selected from the group consisting of: halogen, (C₁-C₆)alkyl, (C₁-C₆)alkoxy, —CN, —CF₃, —OCF₃, —NRₐR_{b}, —Si(R_{c})₃, —BRₐR_{b}, (C₂-C₆)alkenyl, (C₂-C₆)alkynyl, —C(═O)—NRₐR_{b}, and —C(═O)—O(C₁-C₆)alkyl, Rₐ, R_{b} and R_{c} being as defined above; and
said step leading to the formation of a compound AH and SCF₂, and
a step of reacting SCF₂ obtained at the previous step with a compound RH, R being a hydrocarbon group, comprising optionally at least one heteroatom,
for obtaining a compound having the formula R—C(═S)—F.

The present invention provides different processes for the valorization of the fluorinated residues in situ generated during a de-fluorination reaction. Those residues might exist under different forms including XSCF₃ (X being any counteranion such as Na or H), such as a HSCF₃, a highly volatile gas (bp=approx. −30 °C), or as a thiocarbonyl fluoride in equilibrium with a fluoride anion. The HSCF₃, or thiocarbonyl fluoride/F- can be trapped for easy disposal, or can react with other compounds to afford another fluorinated molecule of interest.

The present invention also relates to a process for trapping a fluorinated residue, such as HSCF₃, or the fluoride anion, comprising the following steps:
a step of reacting a compound having the formula (I)

A-SCF₃ (I)
with a nucleophile, in the presence of a metallic complex comprising nickel, a ligand, and a solvent,
A being as defined above,
said step leading to the release of a fluorinated residue, such as HSCF₃, or the fluoride anion, and
a step of reacting said fluorinated residue, such as HSCF₃, or the fluoride anion obtained at the previous step with a compound RH, R being a hydrocarbon group, comprising optionally at least one heteroatom.

The following definitions are set forth to illustrate and define the meaning and scope of the various terms used to describe the invention herein.

The expression “Cₜ-C_{z}” means a carbon-based chain which can have from t to z carbon atoms, for example C₁-C₃ means a carbon-based chain which can have from 1 to 3 carbon atoms.

The term “alkyl group” means: a linear or branched, saturated, hydrocarbon-based aliphatic group comprising, unless otherwise mentioned, from 1 to 12 carbon atoms. By way of examples, mention may be made of methyl, ethyl, n-propyl, isopropyl, butyl, isobutyl, tert-butyl or pentyl groups.

The term “aryl group” means: a cyclic aromatic group comprising between 6 and 10 carbon atoms. By way of examples of aryl groups, mention may be made of phenyl or naphthyl groups.

The term “heteroaryl group” means: a 5- to 10-membered aromatic monocyclic or bicyclic group containing from 1 to 4 heteroatoms selected from O, S or N. By way of examples, mention may be made of imidazolyl, thiazolyl, oxazolyl, furanyl, thiophenyl, pyrazolyl, oxadiazolyl, tetrazolyl, pyridinyl, pyrazinyl, pyrimidinyl, pyridazinyl, indolyl, benzofuranyl, benzothiophenyl, benzoxazolyl, benzimidazolyl, indazolyl, benzothiazolyl, isobenzothiazolyl, benzotriazolyl, quinolinyl and isoquinolinyl groups.

By way of a heteroaryl comprising 5 to 6 atoms, including 1 to 4 nitrogen atoms, mention may in particular be made of the following representative groups: pyrrolyl, pyrazolyl, 1,2,3-triazolyl, 1,2,4-triazolyl, tetrazolyl and 1,2,3-triazinyl. Mention may also be made, by way of heteroaryl, of thiophenyl, oxazolyl, furazanyl, 1,2,4-thiadiazolyl, naphthyridinyl, quinoxalinyl, phthalazinyl, imidazo[1,2-a]pyridine, imidazo[2,1-b]thiazolyl, cinnolinyl, benzofurazanyl, azaindolyl, benzimidazolyl, benzothiophenyl, thienopyridyl, thienopyrimidinyl, pyrrolopyridyl, imidazopyridyl, benzoazaindole, 1,2,4-triazinyl, indolizinyl, isoxazolyl, isoquinolinyl, isothiazolyl, purinyl, quinazolinyl, quinolinyl, isoquinolyl, 1,3,4-thiadiazolyl, thiazolyl, isothiazolyl, carbazolyl, and also the corresponding groups resulting from their fusion or from fusion with the phenyl nucleus.

When an alkyl group is substituted with an aryl group, the term “arylalkyl” or “aralkyl” group is used. The “arylalkyl” or “aralkyl” groups are aryl-alkyl-groups, the aryl and alkyl groups being as defined above. Among the arylalkyl groups, mention may in particular be made of the benzyl or phenethyl groups.

The term “halogen” means: a fluorine, a chlorine, a bromine or an iodine.

The term “alkoxy group” means: an —O-alkyl group where the alkyl group is as previously defined. By way of examples, mention may be made of —O—(C₁-C₄)alkyl groups, and in particular the —O-methyl group, the —O-ethyl group, as —O—C₃alkyl group, the —O-propyl group, the —O-isopropyl group, and as —O—C₄alkyl group, the —O-butyl, —O-isobutyl or —O-tert-butyl group.

The term “alkynyl” as employed herein includes unsaturated, nonaromatic, hydrocarbon groups having 2 to 6 carbons, and comprising at least one triple bond. Preferably, the alkynyl group is linear. Preferably, the alkynyl group is a —(CH₂)ₘ—C≡CH group, m being an integer comprised from 1 to 4.

The term “alkenyl” as employed herein includes unsaturated, nonaromatic, hydrocarbon groups having 2 to 6 carbons, and comprising at least one double bond. Preferably, the alkenyl group is linear. Preferably, the alkenyl group is a —(CH₂)ₘCH═CH₂ group, m being an integer comprised from 1 to 4.

The abovementioned “alkyl”, “aryl”, and “heteroaryl” groups can be substituted with one or more substituents. Among these substituents, mention may be made of the following groups: amino, hydroxyl, thiol, oxo, halogen, alkyl, alkoxy, alkylthio, alkylamino, aryloxy, arylalkoxy, cyano, trifluoromethyl, carboxy or carboxyalkyl.

The term “alkylthio” means: an —S-alkyl group, the alkyl group being as defined above.

The term “alkylamino” means: an —NH-alkyl group, the alkyl group being as defined above.

The term “aryloxy” means: an —O-aryl group, the aryl group being as defined above.

The term “arylalkoxy” means: an aryl-alkoxy-group, the aryl and alkoxy groups being as defined above.

The term “carboxyalkyl” means: an HOOC-alkyl-group, the alkyl group being as defined above. As examples of carboxyalkyl groups, mention may in particular be made of carboxymethyl or carboxyethyl.

The term “haloalkyl group” means: an alkyl group as defined above, in which one or more of the hydrogen atoms is (are) replaced with a halogen atom. By way of example, mention may be made of fluoroalkyls, in particular CF₃ or CHF₂.

The term “carboxyl” means: a COOH group.

The term “oxo” means: “═O”.

In one embodiment, the compound RH is selected from the group consisting of: amines, alcohols, thiols, phosphines, phosphonates, Grignard reagents, R′Li, selenols, R′₂B(OH), and R′₃SiOH, R′ representing an optionally substituted alkyl, aryl, vinyl, or alkynyl group, said alkyl, aryl, and alkynyl groups being as defined above.

In one embodiment, the compound RH is selected from the group consisting of: secondary alkyl amines, anilines, alkyl alcohols, alkyl thiols, aryl thiols, aryl selenols, phosphine oxides, aryl phosphines, and Grignard reagents.

Preferably, the compound RH is selected from the group consisting of: HNR_{d}Rₑ, and OR_{f}, R_{d} and Rₑ being independently selected from the group consisting of: (C₁-C₆)alkyl groups and (C₆-C₁₀)aryl groups; and R_{f} being a (C₁-C₆)alkyl group or a (C₆-C₁₀)aryl(C₁-C₆)alkyl group.

In one embodiment of the process according to the invention, the nucleophile is an hydride.

In one embodiment of the process according to the invention, the nucleophile is a silane compound of formula R²(R³)₂SiH, R² and R³ being independently from each other selected from (C₁-C₆)alkyl, (C₁-C₆)alkoxy, and (C₆-C₁₀)aryl groups, said aryl groups being optionally substituted with at least one substituent selected from: (C₁-C₆)alkyl, (C₁-C₆)alkoxy, —CN, —OCF₃, —NRₐR_{b}, —Si(R_{c})₃, —BRₐR_{b}, (C₂-C₆)alkenyl, (C₂-C₆)alkynyl, —C(═O)—NRₐR_{b}, —C(═O)—O(C₁-C₆)alkyl, and CF₃, Rₐ, R_{b} and R_{c} being as defined above.

In one embodiment of the process according to the invention, the nucleophile is a silane compound of formula R²(R³)₂SiH, R² and R³ being independently from each other selected from (C₁-C₆)alkyl, (C₁-C₆)alkoxy, and (C₆-C₁₀)aryl groups, said aryl groups being optionally substituted with at least one substituent selected from: (C₁-C₆)alkyl, (C₁-C₆)alkoxy, and —C(═O)—O(C₁-C₆)alkyl, and CF₃.

In one embodiment of the process according to the invention, the nucleophile is selected from the group consisting of: EtMe₂SiH, Me(OMe)₂SiH, Me(OEt)₂SiH, (OEt)₃SiH, Et₃SiH, tBuMe₂SiH, Me₂PhSiH, Me₂BnSiH, MePh₂SiH, Ph₃SiH, Me₂(p-CF₃(C₆H₄))SiH, Me₂(p-tBu(C₆H₄))SiH and 1,1,3,3-tetramethyldisiloxane (TMDSO).

In one embodiment, the nucleophile is Me₂(p-CF₃(C₆H₄))SiH or Me₂BnSiH, and preferably the nucleophile is Me₂(p-CF₃(C₆H₄))SiH.

In one embodiment of the process according to the invention, the nucleophile is selected from the group consisting of: alkyl amines, cycloalkyl amines, aryl amines, alkyl thiols, thiophenol compounds, aryl selenol compounds, phosphine oxides, phosphines, Grignard reagents, alcohols or esters. The alkyl amines, cycloalkyl amines and aryl amines can be primary and/or secondary amines.

In particular, the nucleophile is a ZH group, wherein Z is selected from the group consisting of: (C₁-C₆)alkyl, —O—(C₁-C₁₂)alkyl; heterocycloalkyl, —S-cyclo(C₃-C₁₀)alkyl, —P(═O)—Z¹Z², and —X¹—Ar¹, X¹—(C₁-C₁₂)alkyl-Het¹, where X¹ is S, Se, NH or N-alkyl, Ar¹ is an optionally substituted (C₆-C₁₀)aryl group, Het¹ is an optionally substituted heteroaryl group and Z¹ and Z² are, independently from each other, a (C₁-C₆)alkyl group or a (C₆-C₁₀)aryl group.

Preferably, Z is selected from the group consisting of: (C₁-C₆)alkyl, heterocycloalkyl, —S-cyclo(C₃-C₁₀)alkyl, —P(═O)—Z¹Z², and —X¹—Ar¹, X¹ being S, Se or NH, and Ar¹ being an optionally substituted (C₆-C₁₀)aryl group, and Z¹ and Z² being, independently from each other, a (C₁-C₆)alkyl group or a (C₆-C₁₀)aryl group.

The terms “alkyl”, “aryl”, “heterocycloalkyl”, heteroaryl” and their optional substituents are as defined above.

More preferably, the nucleophile is selected from the group consisting of: heterocycloalkyl groups comprising at least one nitrogen and/or sulfur atom such as morpholine, aniline groups and derivatives thereof, such as 4-methylaniline,
H—S-(cyclo)alkyl groups, such as CySH, H—S-aryl groups, such as 4-methylthiophenol,
H—Se-aryl groups, such as benzene selenol, phosphine oxides such as HP(═O)Ph₂, phosphines such as HPPh₂ and Grignard reagents such as Alk-MgBr, in particular iPrMgBr.

According to one embodiment, when the nucleophile is selected from the group consisting of: alkyl amines, cycloalkyl amines, aryl amines, alkyl thiols, thiophenol compounds, aryl selenol compounds, phosphine oxides, phosphines, Grignard reagents, alcohols or esters, the process of the invention as defined above can further comprise the use of a hydride compound, such as LiHMDS. In this embodiment, the compound A-SCF₃ of formula (I) is reacted with the nucleophile in the presence of the metallic complex, the ligand, and the solvent, and also with a hydride compound.

In this embodiment, the nucleophile is, in particular, a Grignard reactant.

According to one embodiment, in the process according to the invention, the ligand is selected from the group consisting of: 1,5-cyclooctadiene (COD), 1,2-Bis(dicyclohexylphosphino)ethane (dcype), 1,1′-Bis(diphenylphosphino)ferrocene (dppf), 1,1′-Bis(di-tert-butylphosphino)ferrocene (DTBPF), 4,5-Bis(diphenyl-phosphino)-9,9-dimethylxanthene (XantPhos), triphenylarsine (AsPh₃), and triphenylphosphane (PPh₃).

The term “ligand” refers to a chemical component that is able to bind to the metal of the metallic complex. The initial state of a metallic complex is the state where the metallic complex is used in the reaction medium. In the initial state of the metallic complex, the ligand can be bound to the metal.

Alternatively, the ligand can be added to the reaction medium in addition to the metallic complex. In such case, the ligand will bind to the metal in the reaction medium.

According to an embodiment of the process, the ligand is bound to the metal in the initial state of the metallic complex.

According to another embodiment of the process, the ligand is added to the reaction medium in addition to the metallic complex.

According to an embodiment of the process, at least one first ligand is bound to the metal in the initial state of the metallic complex and at least one second ligand is added to the reaction medium in addition to the metallic complex, wherein the first and second ligands are identical or different.

Preferably, the metallic complex is a Ni(0) complex such as bis(1,5-cyclooctadiene)nickel (Ni[cod]₂) or bis(triphenylphosphine)nickel (Ni[PPh₃]₂).

According to another embodiment, the metallic complex comprises nickel, palladium or rhodium(I). For example, the metallic complex can be a Pd(0) complex such as tris(dibenzylideneacetone)dipalladium(0) of formula Pd₂dba₃ or tetrakis(triphenylphosphine)palladium(0) of formula Pd(PPh₃)₄. In particular, the metallic complex can be a Pd(II) complex such as, for example, [1,3-Bis(2,6-Diisopropylphenyl)imidazol-2-ylidene](3-chloropyridyl)palladium(II) dichloride (PEPSI Pd IPr, 905459-27-0), palladium(II) acetate of formula Pd(OAc)₂ or allylpalladium (II) chloride dimer of formula [Pd(π-allyl)Cl]₂. Alternatively, the metallic complex can be a Rh(I) complex such as, for example, rhodium carbonyl chloride of formula [Rh(CO)₂Cl]₂ or a hydroxy(cyclooctadiene)rhodium(I) dimer of formula [Rh(OH)COD]₂.

According to one embodiment, the solvent used in the reaction of the compound of formula (I) with the nucleophile according to the invention is selected from the usual solvents used in the field. Preferably, the solvent for this step according to the invention is toluene.

According to one embodiment of the process according to the invention, the reaction of the compound of formula (I) with the nucleophile is carried out a temperature from 90 °C to 140 °C. Preferably, this reaction may be carried out at 90 °C, 110 °C, 120 °C, and 140 °C.

According to one embodiment, in formula (I) as defined above, A is selected from the heteroaryl groups comprising from 5 to 10 atoms and including at least one heteroatom selected from O, N, and S, said heteroaryl groups being optionally substituted with at least one substituent selected from: halogen, (C₁-C₆)alkyl, (C₁-C₆)alkoxy, —CN, —CF₃, —OCF₃, —NRₐR_{b}, —Si(R_{c})₃, —BRₐR_{b}, (C₂-C₆)alkenyl, (C₂-C₆)alkynyl, —C(═O)—NRₐR_{b}, and —C(═O)—O(C₁-C₆)alkyl, Rₐ, R_{b} and R_{c} being as defined above.

The present invention also relates to the process as defined above, wherein the compound of formula (I) is a compound having the following formula (I-1):

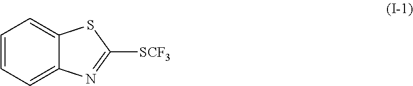

### EXAMPLES

### Example 1: Process for Trapping Thiocarbonyl Fluoride in the Presence of Secondary Anilines as Compound RH

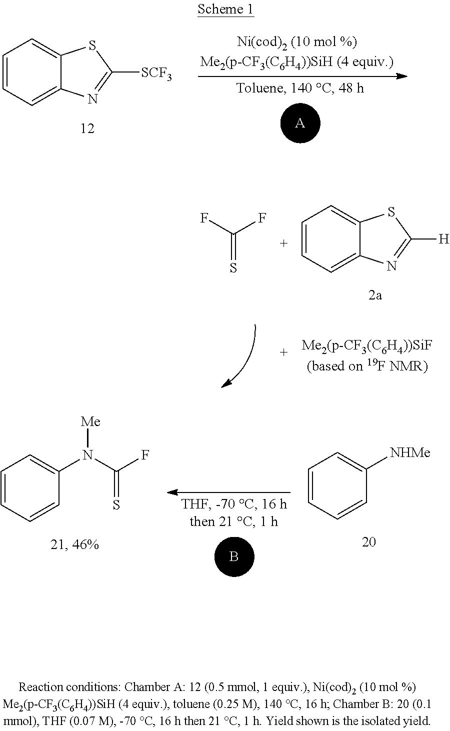

An oven-dried two-chamber tubes both equipped with a stirring bar (chamber A: mL and chamber B: 2 mL) connected by a reflux condenser was filled as follow: Ni (cod)₂ (14 mg, 0.05 mmol, 10 mol %), 2-((trifluoromethyl)thio)benzo[d]thiazole 12 (118 mg, 0.5 mmol, 1 equiv.), Me₂(p-CF₃(C₆H₄))SiH (408 mg, 2 mmol, 4 equiv.) and toluene (2 mL) under argon for the chamber A and with secondary aniline (0.1 mmol) and THF (1.5 mL) under argon for the chamber B. The resulting solutions were stirred at 140 °C for 16 h (chamber A) and at −70 °C for 16 h then at 21 °C for 1 h (chamber B). The residue of the chamber B was concentrated under vacuum and purified by silica gel flash column chromatography to afford the desired products.

### Example 2: Process for Trapping Thiocarbonyl Fluoride in the Presence of Alkyl Alcohols as RH

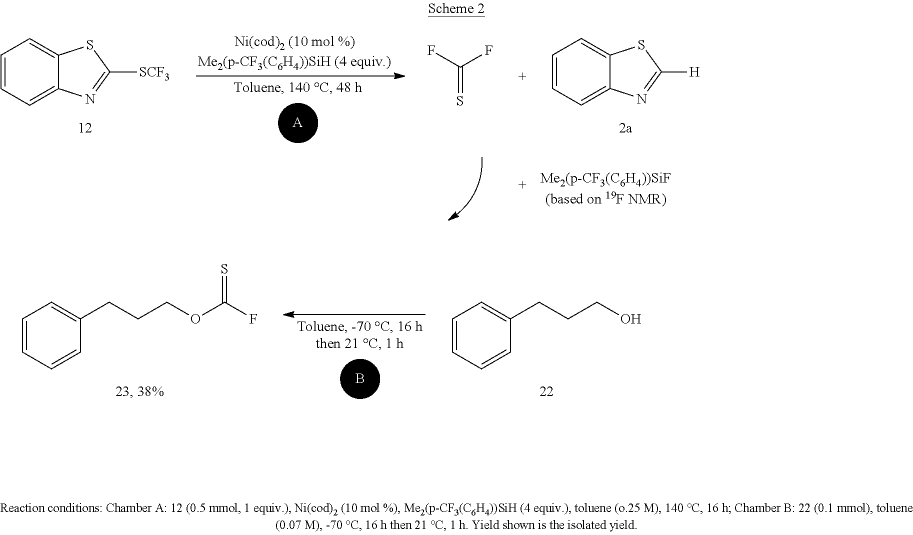

An oven-dried two-chamber tubes both equipped with a stirring bar (chamber A: 10 mL and chamber B: 2 mL) connected by a reflux condenser was filled as follow: Ni (cod)₂ (14 mg, 0.05 mmol, 10 mol %), 2-((trifluoromethyl)thio)benzo[d]thiazole 12 (118 mg, 0.5 mmol, 1 equiv.), Me₂(p-CF₃(C₆H₄))SiH (408 mg, 2 mmol, 4 equiv.) and toluene (2 mL) under argon for the chamber A and with alkyl alcohol (0.1 mmol) and toluene (1.5 mL) under argon for the chamber B. The resulting solutions were stirred at 140 °C for 16 h (chamber A) and at −70 °C for 16 h then at 21 °C for 1 h (chamber B). The residue of the chamber B was concentrated under vacuum and purified by silica gel flash column chromatography by dry loading the samples and eluting with a solvent system as noted below to afford the desired products.

### Example 3: Step of Reacting the Compound of Formula (I-1) in the Presence of Various Nucleophiles, Metallic Complexes and Ligands

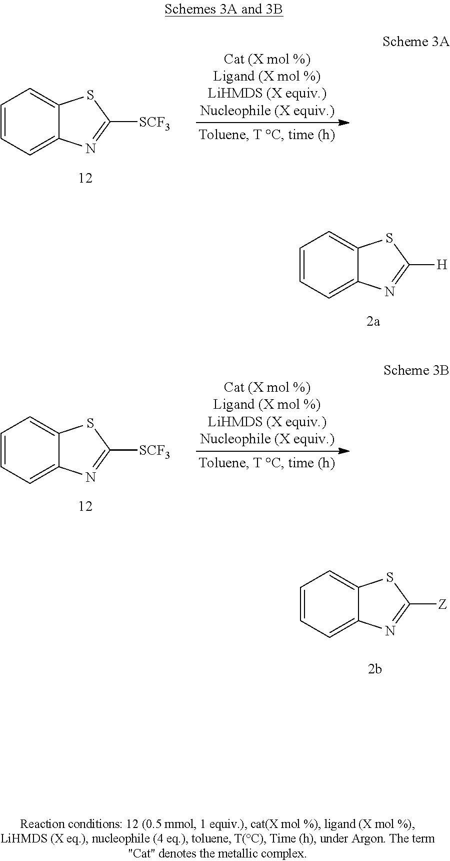

An oven-dried 10 mL tube equipped with a stirring bar was charged with a metallic complex (cat, X mol %), a ligand (X mol %), LiHMDS (X eq.), 2-((trifluoromethyl)thio)benzo[d]thiazole 12 (118 mg, 0.5 mmol, 1 eq.), a nucleophile (4 eq), and toluene under argon. The resulting solution was stirred at a Temperature T (°C) for 16 h or 48 h. The reaction conditions are reported in Table 1 below. The mixture was allowed to cool down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The residue was purified by silica gel flash column chromatography to afford the desired product 2a and 2c-2i. The corresponding yields are reported in Scheme 3C and Table 1 below.

TABLE 1

| | Cat | Nucleophile | LiHMDS | Ligand | T | Time | Yield |
|---|---|---|---|---|---|---|---|
| Product | (X mol %) | (4 eq.) | (X eq) | (X mol %) | (°C) | (h) | (%) |
| 2a | Ni(cod)₂ | Me₂(p- | — | — | 140 | 48 | 69 |
| | (10 mol %) | CF₃(C₆H₄)) | | | | | |
| | | SiH | | | | | |
| | | (4 eq), | | | | | |
| 2a | Ni(cod)₂ | Me₂PhSiH | — | — | 140 | 16 | 66 |
| | (10 mol %) | (4 eq.) | | | | | |
| 2a | Ni(cod)₂ | Me₂(p- | — | XantPhos | 140 | 48 | 48 |
| | (10 mol %) | CF₃(C₆H₄)) | | | | | |
| | | SiH | | | | | |
| | | (4 eq) | | | | | |
| 2c | Ni(cod)₂ | Morpholine | 5 eq | dcype | 100 | 16 | 71 |
| | (5 mol %) | (4 eq) | | (5 mol %) | | | |
| 2c | Ni(PPh₃)₂ | Morpholine | 5 eq | Dcype (5 | 100 | 16 | 47 |
| | | (4 eq) | | mol %) | | | |
| 2c | Ni(PPh₃)₂ | Morpholine | 5 eq | — | 100 | 16 | 53 |
| | | (4 eq) | | | | | |
| 2d | PEPSI Pd | 4- | 5 eq | — | 100 | 16 | 48 |
| | IPr | methylaniline | | | | | |
| | (5 mol %) | (4 eq) | | | | | |
| 2d | PEPSI Pd | 4- | 5 eq | dcype | 100 | 16 | 48 |
| | IPr | methylaniline | | (5 mol %) | | | |
| | (5 mol %) | (4 eq) | | | | | |
| 2e | Ni(cod)₂ | N- | 5 eq | dcype | 100 | 16 | 94 |
| | (5 mol %) | Methylaniline | | (5 mol %) | | | |
| | | (4 eq) | | | | | |
| 2f | Ni(cod)₂ | Desipramine | 5 eq | dcype | 100 | 16 | 86 |
| | (5 mol %) | (4 eq) | | (5 mol %) | | | |
| 2g | Ni(cod)₂ | 4-Methyl- | 5 eq | dcype | 100 | 16 | 75 |
| | (5 mol %) | benzenethiol | | (5 mol %) | | | |
| | | (4 eq) | | | | | |
| 2h | Ni(cod)₂ | HPPh₂ | 5 eq | dcype | 100 | 16 | 63 |
| | (5 mol %) | (4 eq) | | (5 mol %) | | | |
| 2i | Ni(cod)₂ | Pentanol | 5 eq | dcype | 100 | 16 | 81 |
| | (5 mol %) | (4 eq) | | (5 mol %) | | | |

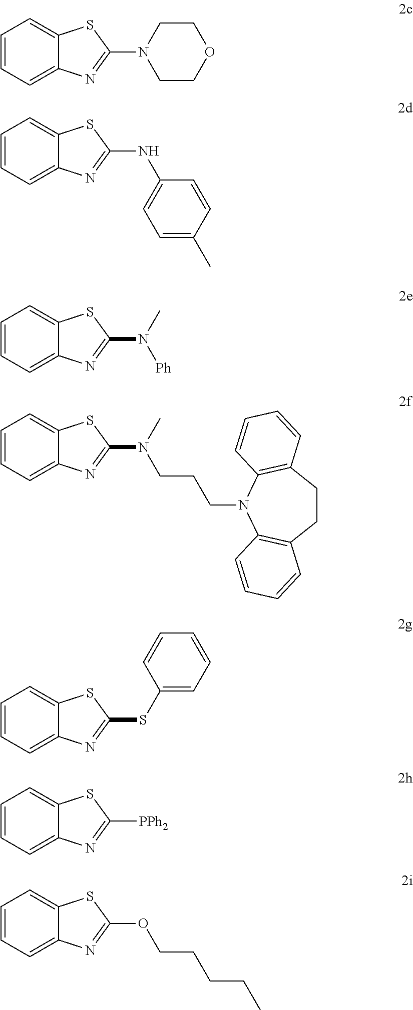

Scheme 3C: Compounds 2c to 2i were synthetized according to scheme 3B in presence of a nucleophile of formula ZH.

The results show that the cleavage of the C—S bond (step leading to the formation of a compound AH and SCF₂ from a compound of formula (I) according to the invention) is achieved with high yields in the presence of various metallic complexes, nucleophiles and ligands. It can be expected that the process of trapping thiocarbonyl fluoride from a A-SCF₃ compound using these reaction conditions will be achieved with good yields.

### Example 4: Step of Reacting a A-SCF₃ Compound, Having a (C₆-C₁₀)Aryl Group as A

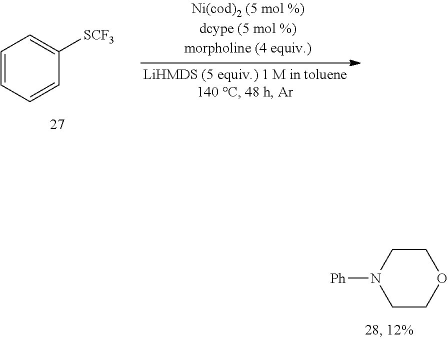

Scheme 4: Reaction conditions: 27 (0.5 mmol, 1 equiv.), Ni (cod)₂ (5 mol %), dcype (5 mol %), LiHMDS 1M solution in toluene (5 equiv.), morpholine (4 equiv.), 140 °C, 48 h, under Argon. Yield shown is the isolated yield.

An oven-dried 10 mL tube equipped with a stirring bar was charged with Ni (cod)₂ (7 mg, 0.025 mmol, 5 mol %), dcype (11 mg, 0.025 mmol, 5 mol LiHMDS in toluene (2.5 mL, 1M, 418 mg, 2.5 mmol, 5 equiv), 27 (0.5 mmol, 1 equiv.) and morpholine (2 mmol, 4 equiv.) under argon. The resulting solution was stirred at 140 °C for 48 h. The mixture was allowed to cool down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The residue was purified by silica gel flash column chromatography to isolate the desired product 28.

This result shows that the cleavage of the C—S bond (step leading to the release of a fluorinated residue) is achieved in a presence of an aryl. It can be expected that the process of trapping thiocarbonyl fluoride from a A-SCF₃ compound, A being an aryl, will be achieved with good yields.

### Example 5: Step of Reacting a A-SCF₃ Compound, Having a Vinyl Group as A

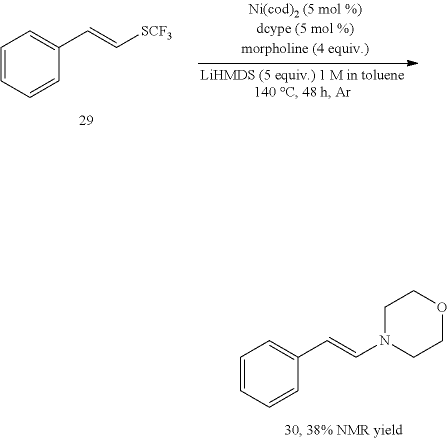

Scheme 5: Reaction conditions: 29 (0.3 mmol, 1 equiv.), Ni (cod)₂ (5 mol %), dcype (5 mol %), LiHMDS 1M solution in toluene (5 equiv.), morpholine (4 equiv.), 140 °C, 48 h, under Argon. Yields were determined by 1H NMR using Nitromethane as an internal standard.

An oven-dried 10 mL tube equipped with a stirring bar was charged with Ni (cod)₂ (4 mg, 0.015 mmol, 5 mol %), dcype (6 mg, 0.015 mmol, 5 mol %), LiHMDS in toluene (1.5 mL, 1M, 251 mg, 1.5 mmol, 5 equiv.), 47 (0.3 mmol, 1 equiv.) and morpholine (1.2 mmol, 4 equiv.) under argon. The resulting solution was stirred at 140 °C for 48 h. The mixture was allowed to cool down to 21 °C. EtOAc (10 mL) was added in the mixture and the resulting solution was washed with an aqueous solution of NaOH (1M, 10 mL). The aqueous phase was extracted with EtOAc (3×20 mL) and the combined organic layers were dried over MgSO₄ and concentrated under vacuum. The yield of 30 was determined by ¹H NMR using Nitromethane as an internal standard.

This result shows that the cleavage of the C—S bond (step leading to the release of a fluorinated residue) is achieved in a presence of a vinyl. It can be expected that the process of trapping thiocarbonyl fluoride from a A-SCF₃ compound, A being an vinyl, will be achieved with good yields.

What is claimed is:

1. A process for trapping SCF₂ comprising the following steps:
a step of reacting a compound having the formula (I)
A-SCF₃ (I)
with a nucleophile, in the presence of a metallic complex comprising nickel, palladium or rhodium(I), a ligand, and a solvent,
A being selected from the group consisting of:
(C₆-C₁₀)aryl groups,
said aryl groups being optionally substituted with at least one substituent preferably selected from the group consisting of: halogen, (C₁-C₆)alkyl, (C₁-C₆)alkoxy, —CN, —CF₃, —OCF₃, —NRₐR_{b}, —SiRₐR_{b}R_{c}, —BRₐR_{b}, (C₂-C₆)alkenyl, (C₂-C₆)alkynyl, —C(═O)—NRₐR_{b}, and —C(═O)—O(C₁-C₆)alkyl;
Rₐ and R_{b} being independently from each other H or a (C₁-C₆)alkyl group;
R_{c} being a (C₁-C₆)alkyl group;
heteroaryl groups comprising from 5 to 10 atoms and including at least one heteroatom selected from O, N, and S,
said heteroaryl groups being optionally substituted with at least one substituent preferably selected from: halogen, (C₁-C₆)alkyl, (C₁-C₆)alkoxy, —CN, —CF₃, —OCF₃, —NRₐR_{b}, —Si(R_{c})₃, —BRₐR_{b}, (C₂-C₆)alkenyl, (C₂-C₆)alkynyl, —C(═O)—NRₐR_{b}, and —C(═O)—O(C₁-C₆)alkyl, Rₐ, R_{b} and R_{c} being as defined above; and
a vinyl compound having the following formula (III): 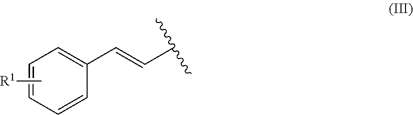
R¹ being selected from the group consisting of: halogen, (C₁-C₆)alkyl, (C₁-C₆)alkoxy, —CN, —CF₃, —OCF₃, —NRₐR_{b}, —Si(R_{c})₃, —BRₐR_{b}, (C₂-C₆)alkenyl, (C₂-C₆)alkynyl, —C(═O)—NRₐR_{b}, and —C(═O)—O(C₁-C₆)alkyl, Rₐ, R_{b} and R_{c} being as defined above; and
said step leading to the formation of a compound AH or a compound AZ, and SCF₂, wherein Z is a hydrocarbon group, optionally including at least one heteroatom such as S, N or O, and/or at least one atom other than C or H, such as Se, Si, B, or P, and
a step of reacting SCF₂ with a compound RH, R being a hydrocarbon group, comprising optionally at least one heteroatom,
for obtaining a compound having the formula R—C(═S)—F.

2. The process according to claim 1, further comprising the following steps:
a step of reacting a compound having the formula (I)
A-SCF₃ (I)
with a nucleophile, in the presence of a metallic complex comprising nickel, a ligand, and a solvent, A being defined as in claim 1; and said step leading to the formation of a compound AH and SCF₂, and
a step of reacting SCF₂ with a compound RH, R being a hydrocarbon group, comprising optionally at least one heteroatom,
for obtaining a compound having the formula R—C(═S)—F.

3. The process according to claim 1, wherein the compound RH is selected from the group consisting of: amines, alcohols, thiols, phosphines, phosphonates, Grignard reagents, R′Li, selenols, R′₂B(OH), and R′₃SiOH, R′ representing an optionally substituted alkyl, aryl, vinyl, or alkynyl group.

4. The process according to claim 1, wherein the compound RH is selected from the group consisting of: secondary alkyl amines, anilines, alkyl alcohols, alkyl thiols, aryl thiols, aryl selenols, phosphine oxides, aryl phosphines, and Grignard reagents.

5. The process according to claim 1, wherein the compound RH is selected from the group consisting of: HNR_{d}Rₑ, and OR_{f}, R_{d} and Rₑ being independently selected from the group consisting of: (C₁-C₆)alkyl groups and (C₆-C₁₀)aryl groups; and R_{f} being a (C₁-C₆)alkyl group or a (C₆-C₁₀)aryl(C₁-C₆)alkyl group.

6. The process according to claim 1, wherein the nucleophile is a silane compound of formula R²(R³)₂SiH, R² and R³ being independently from each other selected from (C₁-C₆)alkyl, (C₁-C₆)alkoxy, and (C₆-C₁₀)aryl groups, said aryl groups being optionally substituted with at least one substituent selected from: (C₁-C₆)alkyl, (C₁-C₆)alkoxy, —CN, —OCF₃, —NRₐR_{b}, —Si(R_{c})₃, —BRₐR_{b}, (C₂-C₆)alkenyl, (C₂-C₆)alkynyl, —C(═O)—NRₐR_{b}, and —C(═O)—O(C₁-C₆)alkyl, and CF₃, Rₐ, R_{b} and R_{c} being as defined in claim 1.

7. The process according to claim 1, wherein the nucleophile is selected from the group consisting of: EtMe₂SiH, Me(OMe)₂SiH, Me(OEt)₂SiH, (OEt)₃SiH, Et₃SiH, tBuMe₂SiH, Me₂PhSiH, Me₂BnSiH, MePh₂SiH, Ph₃SiH, Me₂(p-CF₃(C₆H₄))SiH, and Me₂(p-tBu(C₆H₄))SiH.

8. The process according to claim 1, wherein the nucleophile is Me₂(p-CF₃(C₆H₄))SiH or Me₂BnSiH.

9. The process according to claim 8, wherein the nucleophile is Me₂(p-CF₃(C₆H₄))SiH.

10. The process according to claim 1, wherein the nucleophile is selected from the group consisting of: alkyl amines, cycloalkyl amines, aryl amines, alkyl thiols, thiophenol compounds, aryl selenol compounds, phosphine oxides, phosphines, Grignard reagents, alcohols and esters.

11. The process according to claim 10, wherein the nucleophile is a ZH group, wherein Z is selected from the group consisting of: (C₁-C₆)alkyl, —O—(C₁-C₁₂)alkyl, heterocycloalkyl, —S-cyclo(C₃-C₁₀)alkyl, —P(═O)—Z¹Z², and —X¹—Ar¹, X¹—(C₁-C₁₂)alkyl-Het¹, where X¹ is S, Se, NH or N-alkyl, Ar¹ being an optionally substituted (C₆-C₁₀)aryl group, Het¹ is an optionally substituted heteroaryl group and Z¹ and Z² are, independently from each other, a (C₁-C₆)alkyl group or a (C₆-C₁₀)aryl group.

12. The process according to claim 10, further comprising the use of a hydride compound.

13. The process according to claim 1, wherein the ligand is selected from the group consisting of: 1,5-cyclooctadiene (COD), 1,2-Bis(dicyclohexylphosphino)ethane (dcype), 1,1′-Bis(diphenylphosphino)ferrocene (dppf), 1,1′-Bis(di-tert-butylphosphino)ferrocene (DTBPF), 4,5-Bis(diphenyl-phosphino)-9,9-dimethylxanthene (XantPhos), triphenylarsine (AsPh₃), and triphenylphosphane (PPh₃).

14. The process according to claim 1, wherein the ligand is bound to the metal in the initial state of the metallic complex and/or the ligand is added to a reaction medium in addition to the metallic complex.

15. The process according to claim 1, wherein the solvent is toluene.

16. The process according to claim 1, wherein the reaction of the compound of formula (I) with a hydride is carried out at a temperature from 90 °C to 140 °C.

17. The process according to claim 1, wherein the metallic complex is a Ni(0) complex.

18. The process according to claim 1, wherein A is selected from the heteroaryl groups comprising from 5 to 10 atoms and including at least one heteroatom selected from O, N, and S, said heteroaryl groups being optionally substituted with at least one substituent selected from: halogen, (C₁-C₆)alkyl, (C₁-C₆)alkoxy, —CN, —CF₃, —OCF₃, —NRₐR_{b}, —Si(R_{c})₃, —BRₐR_{b}, (C₂-C₆)alkenyl, (C₂-C₆)alkynyl, —C(═O)—NRₐR_{b}, and —C(═O)—O(C₁-C₆)alkyl, Rₐ, R_{b} and R_{c} being as defined in claim 1.

19. The process according to claim 1, wherein the compound of formula (I) is a compound having the following formula (I-1):

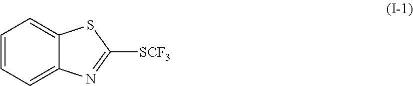

20. The process according to claim 12, wherein the hydride compound is LiHMDS.
